(19) United States
(12) Patent Application Publication
(10) Pub. No.: US 2012/0301436 A1
(43) Pub. Date: Nov. 29, 2012

(54) POLYELECTROLYTE COMPLEX GELS AND SOFT TISSUE AUGMENTATION IMPLANTS COMPRISING THE SAME

(75) Inventors: JEN-CHANG YANG, TAIPEI CITY (TW); SHENG-YANG LEE, TAIPEI CITY (TW); HAW-MING HUANG, TAIPEI CITY (TW); DIAN-YU JI, TAIPEI CITY (TW); HONG-DA WU, TAIPEI CITY (TW); CHEN-FENG MA, TAIPEI CITY (TW)

(73) Assignee: TAIPEI MEDICAL UNIVERSITY, TAIPEI CITY (TW)

(21) Appl. No.: 13/117,477

(22) Filed: May 27, 2011

(51) Int. Cl. A61K 35/12; A61P 13/00; A61K 38/02; A61K 38/18; A61K 47/42

(52) U.S. Cl. 424/93.7; 514/9.6; 514/9.1; 514/8.4; 514/773; 514/1.1

(57) ABSTRACT

The invention provides a polyelectrolyte complex gel comprising a chitosan and a γ-polyglutamic acid (γ-PGA) having a molecular weight from about 1 kDa to about 400 kDa or the salt thereof, wherein the chitosan and the γ-PGA are swollen with an aqueous solution. Also provided is a soft tissue augmentation implant, comprising a polyelectrolyte complex gel of the invention as a carrier or a filler and an optional additive. The polyelectrolyte complex gel and the soft tissue augmentation implant containing the same has long degradation time and better supportability so as to provide good maintenance for soft tissue.

### FIELD OF THE INVENTION

The invention relates to a complex gel useful in soft tissue augmentation and a soft tissue augmentation implant containing the same. In particular, the complex gel is a polyelectrolyte complex (PEC) gel comprising a chitosan and a γ-polyglutamic acid (γ-PGA).

### BACKGROUND OF THE INVENTION

Augmentation of the skin can be an important factor in recovery from injury or for cosmetic or supporting purposes. For example, with normal aging, skin may become loose or creases can form, such as nasal-labial folds, wrinkles, pitting and defects. Creases or lines in the face may adversely affect a person's self esteem or even career. Soft tissue augmentation to correct defects and counteract the effects of aging is becoming increasingly important. Currently, soft tissue augmentation may be achieved by the use of such materials as waxy material, collagen, fat, silicone, poly-lactic acid, polyethylene, polytetrafluoroethylene, or hydrogel based polymer compositions. These materials can be in various forms depending on the use; for example, they can be in the form of thick solution, gel, bead or suspension and used as implants or carriers for delivering the implants. The ideal material for soft tissue augmentation should be sufficiently durable and remain in position and should not migrate from the implantation site.

U.S. Pat. No. 4,803,075 discloses an injectable implant composition for soft tissue augmentation that comprises an aqueous suspension of a particulate biocompatible material such as cross-linked collagen and a biocompatible fluid lubricant such as glycogen or maltose to improve the injectability of the biomaterial suspension. However, the compositions of the patent do not have sufficient durability, so they cannot stay in body for a sufficiently long time.

U.S. Pat. No. 6,537,574 relates to a biocompatible material comprising a matrix of smooth, round, finely divided, and substantially spherical particles of a biocompatible ceramic material close to or in contact with each other which provide a scaffold or lattice for autogenous, three dimensional, randomly oriented, non-scar soft tissue growth at the augmentation site. In one embodiment, after a sterilized sample of 20-45 μm or 75-125 μm of particulate calcium hydroxyapatite suspended in carboxymethylcellulose is injected to tissues, carboxymethylcellulose is absorbed within three months while calcium hydroxyapatite remains in the tissues. Although the implantation of the sample into soft tissues is not difficult, a second injection should be made within three months.

U.K. Patent No. 2,222,176 provides an improved micro-implantation method and apparatus for filling depressed scars, unsymmetrical orbital floors, and superficial bone defects. The patent employs textured micro particles having an outside diameter between about 20 and 3000 microns which may be injected with an appropriate physiologic vehicle and hypodermic needle and the syringe into a predetermined locus, for example, into the base of depressed scars, beneath the skin in areas of depression and beneath the perichondrium or periosteum in surface irregularities of bone and cartilage. In the instances wherein the requirement is for hard substances, biocompatible materials such as certain calcium salts including hydroxyapatite or other such crystalline materials, biocompatible ceramics, biocompatible metals such as certain stainless steel particles, or glass may be utilized. In certain instances, it may be desirable to employ a totally inert vehicle such as silicone oils, fats and esters of hylauranic acids such as ethyl hylauranodate and polyvinylpyrrolidone. However, these vehicles will degrade quickly, so they cannot support targets for a long term.

U.S. Pat. No. 5,344,452 relates to an alloplastic implant based on a histocompatible solid. While the implant is particularly used to even out skin irregularities, it can also be used for any other purpose in plastic surgery. The patent provides particles of 20-40 μm polymethylmethacrylate (PMMA) suspended in collagen which have been inserted into the body, are encapsulated by a delicate capsule of connective tissue or are embedded into connective-tissue fibers, and which remain stationary in the tissue. However, since the collagen is from an animal such as cow, this implant may cause allergy.

U.S. Patent Publication No. 20080025950 A1 discloses compounds such as macromolecules that have been modified in order to facilitate crosslinking by introduction of at least one hydrazide-reactive group and/or aminooxy-reactive group, and methods of making and using thereof for scar-free wound healing, delivering bioactive agents or living cells, preventing adhesion after a surgical procedure or bone and cartilage repair. However, cross-linking reaction must be used to form modified macromolecules.

R. A. Appell, “The Artificial Urinary Sphincter and Periurethral Injections,” Obstetrics and Gynecolocy Report Vol. 2, No. 3, pp. 334-342, (1990), is a survey article disclosing various means of treating urethral sphincteric incompetence, including the use of injectable fillers such as polytetrafluoroethylene micropolymer particles of about 4 to 100 microns in size in irregular shapes, with glycerin and polysorbate. Another periurethral injectable means consists of highly purified bovine dermal collagen that is crosslinked with glutaraldehyde and dispersed in phosphate-buffered physiologic saline. Kresa et al, “Hydron Gel Implants in Vocal Cords,” Otolaryngolocy Head and Neck Surgery, Vol. 98. No. 3, pp. 242-245, (March 1988), discloses a method for treating vocal cord adjustment where there is insufficient closure of the glottis which comprises introducing a shaped implant of a hydrophilic gel that has been previously dried to a glassy, hard state, into the vocal cord. In the above two references, in vivo degradation time is an important factor in evaluating a soft tissue implant. For example, collagen quickly undergoes proteolytic degradation within the body, resulting in relatively short clinical effectiveness. Patients must receive additional injections to maintain tissue reformation, usually at an interval of every few months. Continual submission to the injection procedure is costly and causes inconvenience, discomfort or perhaps pain, and other side effects. As with any invasive medical procedure, injection carries with it the risk of cross-contamination and infection. Therefore, there still exists a need in the art for longer-lasting injectable materials for soft tissue augmentation.

### SUMMARY OF THE INVENTION

The invention provides a polyelectrolyte complex gel comprising a chitosan, a γ-polyglutamic acid (γ-PGA) having a molecular weight from about 1 kDa to about 400 kDa or the salt thereof and an aqueous solution, wherein the chitosan and the γ-PGA are swollen with the aqueous solution.

The invention also provides a soft tissue augmentation implant comprising a polyelectrolyte complex gel of the invention and a carrier or a filler and an optional additive.

### BRIEF DESCRIPTION OF THE DRAWINGS

FIG. 1 shows in vitro dissolution test of the polyelectrolyte complex gel of the invention.

FIG. 2 shows injectability test of the polyelectrolyte complex gel/implant of the invention.

FIG. 3 shows tissue section plots of the polyelectrolyte complex gel/implant of the invention and carrier/implant known in the art.

FIG. 4 shows within 180 days after implantation, the changes of volume retention of the polyelectrolyte complex gel based implant of the invention and implant known in the art.

FIG. 5 shows within 180 days after implantation, the changes of height retention of the polyelectrolyte complex gel based implant of the invention and implant known in the art.

### DETAILED DESCRIPTION OF THE INVENTION

The invention utilizes the cross-linking of oppositely charged polyelectrolytes such as polysaccharide and polypeptide to form a polyelectrolyte complex gel useful in soft tissue augmentation. The invention mixes oppositely charged polyelectrolytes to form a polyelectrolyte complex gel without utilizing chemical crosslinkers. Typical dermal implant system contains at least a filler, a carrier, and an optional additive. Accordingly, the invention develops a polyelectrolyte complex gel that can be used as a carrier or a filler for soft tissue augmentation, depending on degree of cross-linking of the gel. Therefore, the polyelectrolyte complex gel can play the role of a carrier or a filler. The polyelectrolyte complex gel of the invention has advantageous ability to carry substances without cell potential toxicity. Particularly, it is suitable for use in soft tissue augmentation through fine needle syringe due to its good injectability. Moreover, the polyelectrolyte complex gel has long degradation time and better supportability and thus provides good maintenance for soft tissue.

As used in this specification and the appended claims, the singular forms “a”, “an”, and “the” include plural references unless the context clearly dictates otherwise.

The term “soft tissue”, as used herein, refers non-skeletal tissue, i.e. exclusive of bones, ligaments, cartilage, spinal disc and fibrous tissue.

The term “soft tissue augmentation” includes, but is not limited to, the following: dermal tissue augmentation; filling of lines, folds, wrinkles, minor facial depressions, cleft lips and the like, especially in face and neck; correction of minor deformities due to aging or disease, including in hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and orbital groove around the eye; breast augmentation; oral cavity augmentation; chin augmentation; cheek and/or nose augmentation; filling of indentations in the soft tissue, dermal or subcutaneous, due to, e.g., overzealous liposuction or other trauma; filling of acne or traumatic scars and rhytids; filling of nasolabial lines, nasoglabellar lines and infraoral lines; and filling used for urethral injection in urinary incontinence (particularly, stress urinary incontinence).

The term “bioresorbable” means capable of being reabsorbed and eliminated from the body.

The term “biocompatible” means physiologically acceptable to a living tissue and organism.

The term “polyelectrolyte complex gel” means neutral polymer-polymer complex gel composed of macromolecules carrying charges of opposite sign causing the macromolecules to be bound together by electrostatic interactions. It can be immediately formed by mixing a solution of a cationic polymer (a polyelectrolyte having positive charges) and a solution of an anionic polymer (a polyelectrolyte having negative charges).

The term “crosslinking” refers to process whereby oppositely charged polyelectrolytes such as polysaccharide and polypeptide form a polyelectrolyte complex gel.

The term “injected”, “injection”, or “injectability” as used herein is intended to include any administration of the polymer composition, such as by injection, infusion, or any other delivery through any annular delivery device to the subject. Injection includes delivery through a tube.

In one aspect, the invention provides a polyelectrolyte complex gel comprising a chitosan, a γ-polyglutamic acid (γ-PGA) having a molecular weight from about 1 kDa to about 400 kDa or the salt thereof and an aqueous solution, wherein the chitosan and the γ-PGA are swollen with the aqueous solution.

In one embodiment, the molecular weight of γ-PGA ranges from about 1 kDa to about 350 kDa, about 1 kDa to about 300 kDa, about 1 kDa to about 250 kDa, about 1 kDa to about 200 kDa, about 1 kDa to about 150 kDa, about 1 kDa to about 100 kDa, about 1 kDa to about 100 kDa, about 1 kDa to about 50 kDa, about 5 kDa to 350 kDa, about 5 kDa to about 300 kDa, about 5 kDa to about 250 kDa, about 5 kDa to about 200 kDa, about 5 kDa to about 150 kDa, about 5 kDa to about 100 kDa, about 5 kDa to about 50 kDa, about 10 kDa to about 400 kDa, about 10 kDa to about 350 kDa, about 10 kDa to about 300 kDa, about 10 kDa to about 250 kDa, about 10 kDa to about 200 kDa, about 10 kDa to about 150 kDa, about 10 kDa to about 100 kDa, about 10 kDa to about 50 kDa, about 50 kDa to about 400 kDa, about 50 kDa to about 350 kDa, about 50 kDa to about 300 kDa, about 50 kDa to about 250 kDa, about 50 kDa to about 250 kDa, about 50 kDa to about 200 kDa, about 50 kDa to about 150 kDa, about 50 kDa to about 100 kDa, about 100 kDa to about 400 kDa, about 100 kDa to about 350 kDa, about 100 kDa to about 300 kDa, about 100 kDa to about 250 kDa, about 100 kDa to about 200 kDa, or about 100 kDa to about 150 kDa, or any mixture thereof.

In another embodiment, the salt of γ-PGA is in the H form or salt form (such as sodium salt, potassium salt, calcium salt or magnesium salt).

In another embodiment, the amounts of the chitosan and γ-PGA or a salt thereof range from about 0.1 wt % to about 10 wt % and about 0.1 wt % to about 20 wt %. Preferably, the amount of the chitosan is about 0.5 wt % to about 10 wt %, about 1 wt % to about 10 wt %, about 2 wt % to about 10%, about 0.5 wt % to about 5 wt %, about 1 wt % to about 5 wt %, about 2 wt % to about 5 wt %. Preferably, the amount of γ-PGA or a salt thereof is about 0.5 wt % to about 20 wt %, about 1 wt % to about 20 wt %, about 1 wt % to about 15 wt %, about 1 wt % to about 10 wt % or about 1 wt % to about 5 wt %.

In another embodiment, chitosan can be created by N-deacetylation of the chitin polymer. It is a linear polysaccharide composed of randomly distributed β-(1-4)-linked D-glucosamine (deacetylated unit) and N-acetyl-D-glucosamine (acetylated unit). The chitosan used in the combination of the invention refers to a native chitosan or its derivatives. Any commercially available chitosan can be used in the invention. Preferably, chitosan has a molecular amount more than 100 kDa. Preferably, the molecular weight of chitosan is in the range of from about 100 kDa to about 2000 kDa, about 100 kDa to about 1500 kDa, about 100 kDa to about 1000 kDa, about 200 kDa to about 2000 kDa, about 200 kDa to about 1500 kDa, about 200 kDa to about 1500 kDa, about 100 kDa to about 700 kDa, about 100 kDa to about 400 kDa, or about 400 kDa to about 700 kDa, or any mixture thereof.

According to the invention, the polyelectrolyte complex gel can be used as a filler carried in a carrier or a carrier holding a filler for use in soft tissue augmentation. The polyelectrolyte complex gel of the invention is formed by cross-linking chitosan and γ-PGA having a molecular weight from about 1 kDa to about 400 kDa or the salt thereof. The flowability of the polyelectrolyte complex gel of the invention is different depending on the degree of cross-linking. When cross-linking chitosan with γ-PGA having higher molecular weight, the formed polyelectrolyte complex gel has less flowability. In this case, the polyelectrolyte complex gel can be used as a filler for soft tissue augmentation. When cross-linking chitosan with γ-PGA having lower molecular weight, the formed polyelectrolyte complex gel has better flowability. In such a case, the polyelectrolyte complex gel can be used as a carrier for soft tissue augmentation.

Any suitable aqueous solution for swelling the polyelectrolyte complex gel of the invention may be utilized. For example, the gel may be swollen with an aqueous solution having a weak acidic pH. Preferably, the pH ranges from 3.0 to 6.8. Preferably, the aqueous solution is aqua or aqueous alcohol. Examples of the aqueous solution include, but are not limited to, aqua, glycerol, isopropyl alcohol, ethanol, and ethylene glycol, or mixtures thereof. Other suitable solvents for the gel carrier will be apparent to one skilled in the art. Surfactants, stabilizers, pH buffers, and other additives may also be useful, as would be obvious to one skilled in the art.

According to the invention, no cross-linking agent is necessary to form the polyelectrolyte complex gel of the invention. The polyelectrolyte complex gel of the invention is formed on the basis of polyelectronic mechanism. The polyelectrolyte complex gel of the invention has sufficient durability, and can stay in body for a sufficiently long time and still have good supporting ability. Preferably, the polyelectrolyte complex gel of the invention can remain in body for at least 2 months, three months, four months, five months, or six months. More preferably, the polyelectrolyte complex gel of the invention can remain in body for at least 6 months. Preferably, the polyelectrolyte complex gel of the invention can remain in body for 2 to 12 months, 3 to 12 months, 4 to 12 months, 5 to 12 months, 6 to 12 months, 7 to 12 months, 8 to 12 months, 9 to 12 months, 10 to 12 months, 2 to 6 months or 4 to 8 months.

In another aspect, the invention provides a soft tissue augmentation implant comprising a polyelectrolyte complex gel of the invention as a carrier or a filler and an optional additive.

As mentioned in the above, the polyelectrolyte complex gel of the invention can be used as a carrier or a filler. Therefore, the polyelectrolyte complex gel of the invention can be used as a carrier for carrying a filler and an optional additive. Alternatively, the polyelectrolyte complex gel of the invention can be used as a filler carried in a carrier. Depending on the role of the polyelectrolyte complex gel of the invention, it can be combined with a carrier or filler and an optional additive to constitute a soft tissue augmentation implant.

In one embodiment, a variety of biocompatible carriers can be used to hold the polyelectrolyte complex gel of the invention as a filler. The choice of suitable carrier will depend on the particle size, the amount of fillers, the size of injection needle and the nature of the fillers. Examples of the carrier include, but are not limited to, Acacia gel, Carbomer copolymer and homopolymer, Carbomer interpolymer, hydrogel, polysaccharide, macrocyclic polycsaccharide, oligosaccharide, starch, acetyl starch, cellulose, cellulose derivatives, methylcellulose, carboxymethylcellulose sodium, carboxymethylcellulose (CMC), ethyl (hydroxyethyl) cellulose (EHEC), ethylcellulose, hydroxypropyl cellulose, hydroxypropyl methylcellulose (HPMC), ethylcellulose, alkyl cellulose, alkoxy cellulose, hydroxy ethyl cellulose, copovidone, povidone, gelatin, Guar gum, hypromellose, hypromellose acetate succinate, maltodextrin, syrup, agar, alamic acid, aluminum monostearate, attapulgite, gellan gum, hypromellose, maltodextrin, pectin, propylene glycol alginate, sodium alginate, calcium alginate, colloidal silicon dioxide, tragacanth, xanthan gum, lecithin, tridobenzene derivatives, iohexyl, iopamidol, iopentol, sucrose, carrageenan, agarose, mannitol, saccharin sodium, sorbitol, cephalin, acetylenic diol, Carbowax, polyorgano sulfonic acid, alkoxylated surfactants, alkylphenol ethoxylates, ethoxylated fatty acids, alcohol ethoxylates, alcohol alkoxylates, polyethylene oxide, poly(propylene oxide), poly(ethylene glycol), poly(propylene glycol), poly vinyl alcohol (PVA) polymer or copolymer, polyacrylamine, poly(vinylcarboxylic acid), polymethacrylic acid, polyacrylic acid polymer or copolymer, poly amino acids, albumin, collagen, fibrin, bioglue, cellulosics, Carbopol, Poloxamer, Pluronic, Tetronics, PEO-PPO-PEO triblocks copolymer, tetrafunctional block copolymer of PEO-PPO condensed with ethylenadiamine, polyHEMA polymer or copolymer, Hypan polymer or copolymer, starch glycolate polymer or copolymer salt, polyoxyalkylene ether, polyvinyl pyridine, polylysine, polyarginine, poly aspartic acid and poly glutamic acid, polytetramethylene oxide, poly(hydroxy ethyl acrylate), poly(hydroxy ethyl methacrylate), methoxylated pectin gels, cellulose acetate phthalate, organic oils, B-glucan, polysorbate, lactic acid ester, caproic acid ester, hyaluronic acid, dextrin, dextran, dextrose, and mixture of the above.

In one embodiment, a variety of biocompatible fillers can be used to be carried in the polyelectrolyte complex gel of the invention as a carrier. Examples of the filler include, but are not limited to, polysaccharides (such as hyaluronic acid (HA)), inorganic salt, collagen, polyalcohols, hydroxyapatite (such as calcium hydroxyapatite), silicone and gelatin, polymethylmethacrylate or poly-L-lactic acid (PLLA), carboxymethylcellulose, cross-linked CMC hydrogel, fat and silk protein.

According to the invention, the inorganic salt is in a particle form and includes, but is not limited to, calcium phosphate particle, calcium silicate particle, calcium carbonate particle, aluminum oxide particle, zirconium oxide particle, hydroxyapatite particle, zirconium oxide containing hydroxyapatite particle, calcium pyrophosphate particle, tetracalcium phosphate particle, tricalcium phosphate particle, octacalcium phosphate particle, fluorapatite (Ca₁₀(PO₄)₆F₂) particle, calcium apatite particle and a mixture thereof. Preferably, the inorganic salt is calcium phosphate particle, zirconium oxide particle, hydroxyapatite particle, zirconium oxide containing hydroxyapatite particle or a mixture thereof. Preferably, the inorganic salt is hydroxyapatite particle, calcium pyrophosphate particle, tetracalcium phosphate particle, tricalcium phosphate particle, octacalcium phosphate particle, fluorapatite (Ca₁₀(PO₄)₆F₂) particle, calcium apatite particle or a mixture thereof. More preferably, the inorganic salt is zirconium oxide containing hydroxyapatite particle. More preferably, the inorganic salt is hydroxyapatite particle. Most preferably, the inorganic salt is calcium phosphate particle.

In one embodiment, an additive that can be optionally used in the soft tissue augmentation implant of the invention may be numerous materials, including but not limited to cells, proteins and bioactive substances.

According to the invention, the bioactive substances can be therapeutic and, for example, promote tissue growth, i.e., growth factors, or act as an antimicrobial. Preferably, the bioactive substance is epidermal growth factor (EGF), fibroblast growth factor (FGF), nerve growth factor (NGF) or a mixture thereof. More preferably, the bioactive substance is EGF. These substances may also be grafted to or absorbed by the particles, and may be of a nature so that they are time-released in the surrounding tissue. Those skilled in the art will recognize the various bioactive substances that may be incorporated into the implant material and their medical value, depending on the application.

According to the invention, the cells may be adipose (fat) cells, embryonic stem cells, mesenchymal stem cells, neural stem cells, preadipocytes, adipose derivated stem cells or dental pulp stem cells.

In certain embodiments of the invention, the soft tissue augmentation implant is injected, e.g., with syringe or orthoscopic devices. These methods are preferred because they are less invasive than other, e.g., surgical, procedures, lessen the risk of infection, discomfort, and complications, and can be easily controlled in amount and location. One skilled in the art will know the various methods of injection. For example, embodiments of the invention having a particle size of about 500 microns may be injected using an 18-gauge syringe. Those embodiments having smaller particles may be injected with higher-gauge needles, e.g., orthoscopically.

In one embodiment of the invention, the soft tissue augmentation implant is injected subcutaneously into an area having a soft tissue contour defect. The amount implanted is in a sufficient amount to at least partially, preferably entirely, remove the defect. Such defect may include, for example, wrinkles and defects in oral cavity, breast, chin, cheek and/or noses.

In another embodiment of the invention, the soft tissue augmentation implant may be used to control incontinence, particularly stress urinary incontinence. Such incontinence may be the result of disease, aging, or neuromuscular degeneration. It may also result from prostate surgery that causes localized damage to the nerves controlling the sphincter surrounding the urethra.

When this soft tissue augmentation implant is implanted into soft tissue, dense, fibrous and flexible tissue forms around and into the porous portion of the implant. This occurs within a few days of implantation. The implant remains inert within the body, and with the newly formed tissue, augments or shapes the soft tissue as desired

Those skilled in the art will recognize that the polyelectrolyte complex gel, soft tissue augmentation implant and methods of the present invention will have various other uses in addition to the above described embodiments. They will appreciate that the foregoing specification and accompanying drawings are set forth by way of illustration and not limitation of the invention. It will further be appreciated that various modifications and changes may be made therein without departing from the spirit and scope of the present invention, which is to be limited solely by the scope of the appended claims.

### EXAMPLE

### Example 1

### Preparation of Polyelectrolyte Complex Gel of the Invention

30 g of chitosan and 40 g of γ-PGA (Mw=10 kDa) were added to 1,000 g of acetic acid solution of 1 wt % in a vessel to quickly mix for 30 seconds. The mixture was titrated to neutral with 10 N NaOH and then mixed for another 30 minutes. The polyelectrolyte complex gel was allowed to set for a minimum of twelve hours.

For a comparative example showing a gel known in the art, a carboxymethyl cellulose (CMC) carrier as Control 1 was prepared in the following manner: 3 g of CMC and 150 g of glycerin were added to 520 ml of water in a vessel to mix for 30 seconds. The mixture was stirred slowly for 12 hours to be homogenous to form the CMC carrier.

### Example 2

### Preparation of Polyelectrolyte Complex Gel of the Invention

20 g of chitosan were added to 1,000 g of acetic acid solution of 0.5 wt % in a vessel, and then 10 g of γ-PGA (Mw=300 kDa) were also added to quickly mix for 30 seconds. The mixture was titrated to neutral with 10 N NaOH and then mixed for another 30 minutes. The polyelectrolyte complex gel was allowed to set for a minimum of twelve hours.

### Example 3

### Preparation of Hydroxyapatite Particles Contained Polyelectrolyte Complex Gel Implants

The polyelectrolyte complex gel of Example 1 and 430 g of hydroxyapatite particles (particle size ranging from 25 to 45 microns) were thoroughly blended, utilizing a low speed mixer, until all the particles were homogenously distributed in 1,000 ml of the gel suspension.

Furthermore, the CMC carrier of Control 1 and 430 g of hydroxyapatite particles (particle size ranging from 25 to 45 microns) were thoroughly blended, utilizing a low speed mixer, until all the particles were homogenously distributed in 1,000 ml of the carrier to form hydroxyapatite particles contained CMC carrier mixture (Control 2 as the comparative example).

Control 3 as the comparative example was a commercial product of Radiesse®, which contained hydroxyapatite particle microspheres suspended in a CMC carrier, and was produced by BioForm Inc.

### Example 4

### Preparation of Poly-L-Lactic Acid (PLLA) Particles Contained Polyelectrolyte Complex Gel Implants

The polyelectrolyte complex gel of Example 1 and 200 g of poly-L-Lactic Acid (PLLA) particles (particle size ranged from 40 to 63 microns) were thoroughly blended, utilizing a low speed mixer, until all the particles were homogenously distributed in 1,000 ml of the gel suspension.

### Example 5

### Preparation of Poly(methyl methacrylate) Particles Contained Polyelectrolyte Complex Gel Implants

The polyelectrolyte complex gel carrier of Example 1 and 127.8 g of poly(methyl methacrylate) particles (particle size ranging from 100 to 180 microns) were thoroughly blended, utilizing a low speed mixer, until all the particles were homogenously distributed in 1000 ml of the gel suspension.

### Example 6

### Preparation of Silk Microparticles Contained Polyelectrolyte Complex Gel Implants

The polyelectrolyte complex gel of Example 1 and 250 g of silk microparticles (particle size ranged from 20 to 45 microns) were thoroughly blended, utilizing a low speed mixer, until all the particles were homogenously distributed in 1,000 ml of the gel suspension.

### Example 7

### Preparation of Polyelectrolyte Complexes Particles Contained Polyelectrolyte Complex Gel Implants

1 g of chitosan was added into 100 ml of acetic acid solution of 1 wt %. 0.3 g of γ-PGA calcium (Mw=1,000 kDa) and 0.2 g CMC were added to 100 ml of deionized water. These two solutions were blended quickly using a homogenizer for about 30 seconds to form polyelectrolyte complex microparticles. The polyelectrolyte complex gel of Example 1 and 250 g of the polyelectrolyte complex microparticles (particle size ranged from 100 to 120 microns) were thoroughly blended, utilizing a low speed mixer, until all the particles were homogenously distributed in 1000 ml of the gel suspension.

### Example 8

### Preparation of Dental Pulp Stem Cells Contained Polyelectrolyte Complex Gel Implants

The polyelectrolyte complex gel of Example 1 and 1 mL of 1×10⁶ dental pulp stem cells were thoroughly blended, utilizing a low speed mixer, until all the cells were homogenously distributed in 1,000 ml of the gel suspension.

### Example 9

### Preparation of Dental Pulp Stem Cells as well as Hydroxyapatite Particles Contained Polyelectrolyte Complex Gel Implants

The polyelectrolyte complex gel and hydroxyapatite particles mixture of Example 3 and 1 ml of 1×10⁶ dental pulp stem cells were thoroughly blended, utilizing a low speed mixer, until all the cells were homogenously distributed in 9 ml of the gel suspension.

Furthermore, a dental pulp stem cell-containing hydroxyapatite/CMC carrier mixture as Control 4 as the comparative example was prepared in the following manner: the hydroxyapatite/CMC carrier mixture of Control 2 and 1 ml of 1×10⁶ dental pulp stem cells were thoroughly blended, utilizing a low speed mixer, until all the cells were homogenously distributed in 9 ml of the gel suspension.

### Example 10

### In Vitro Dissolution Test of Polyelectrolyte Complex Gel of the Invention

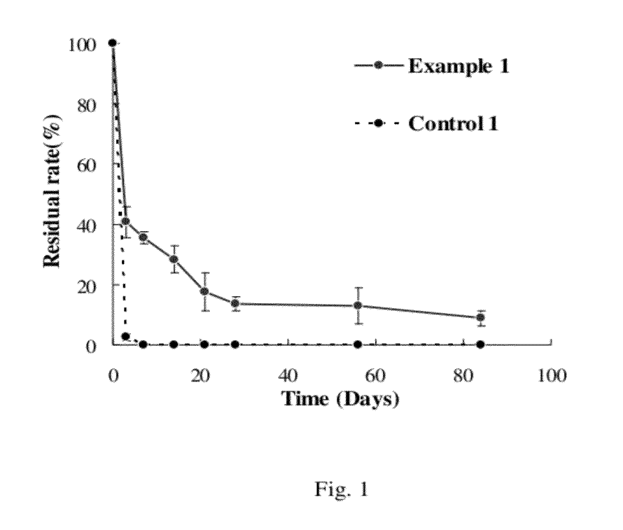

The polyelectrolyte complex gel of Example 1 and Comparative Control 1 were treated with frozen drying to get the dry powder. Powder samples weighing 0.5 g were placed in 40 ml of buffer solution (100 mM NaCl, 45 mM NaHCO₃, 2 mM K₂CO₃), and were taken out after 3 days, and 1, 2, 4, 8 and 12 weeks. Samples were washed with water, filtered, dried and weighed to calculate the sample residual rate ((the original weight−the weight after dissolution)/the original weight). The result was shown in FIG. 1, which shows that the degradation time of Example 1 was longer than that of Control 1, which decreased to below 2.5% after 3 days and almost vanished after 1 week.

### Example 11

### Injectability Test

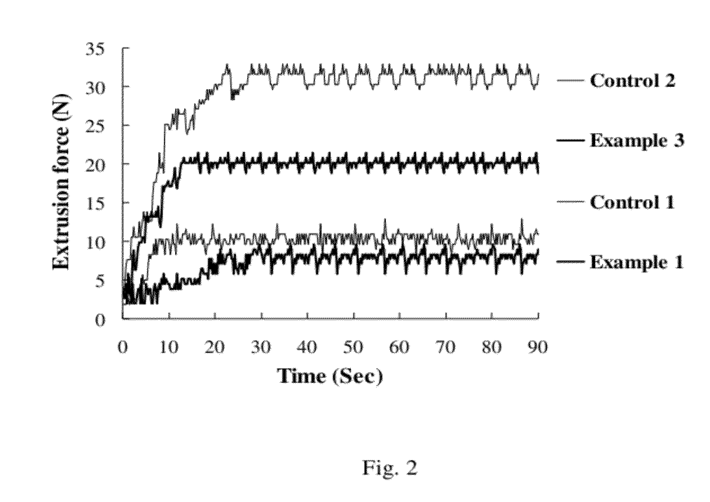

The polyelectrolyte complex gel based implant of Example 1 and Example 3 were transferred into a 3 mL syringe fitted with a 27 G needle. The injectability test was measured on a Texture analyzer (TA.XT Plus, Texture Technologies Corp. UK) at a speed of 15 mm/min and holding for 90 seconds continuously to obtain the extrusion force data. The result was shown in FIG. 2, indicating that the gel based implant of the Examples 1 and 3 have good injectability.

### Example 12

### Animal Study

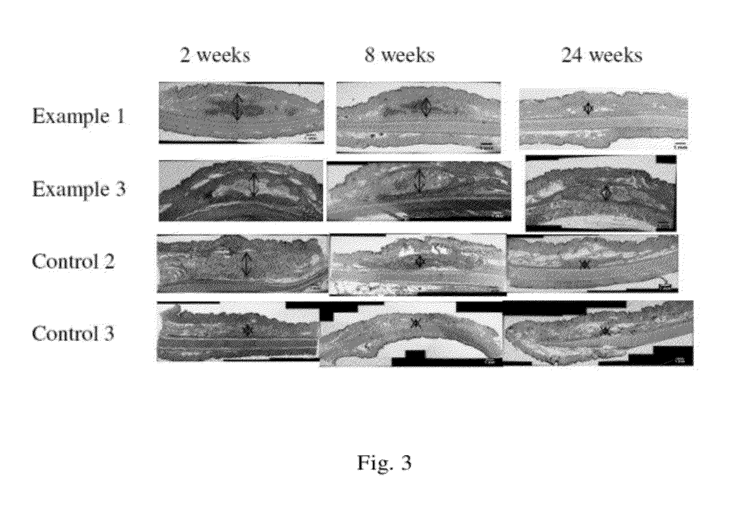

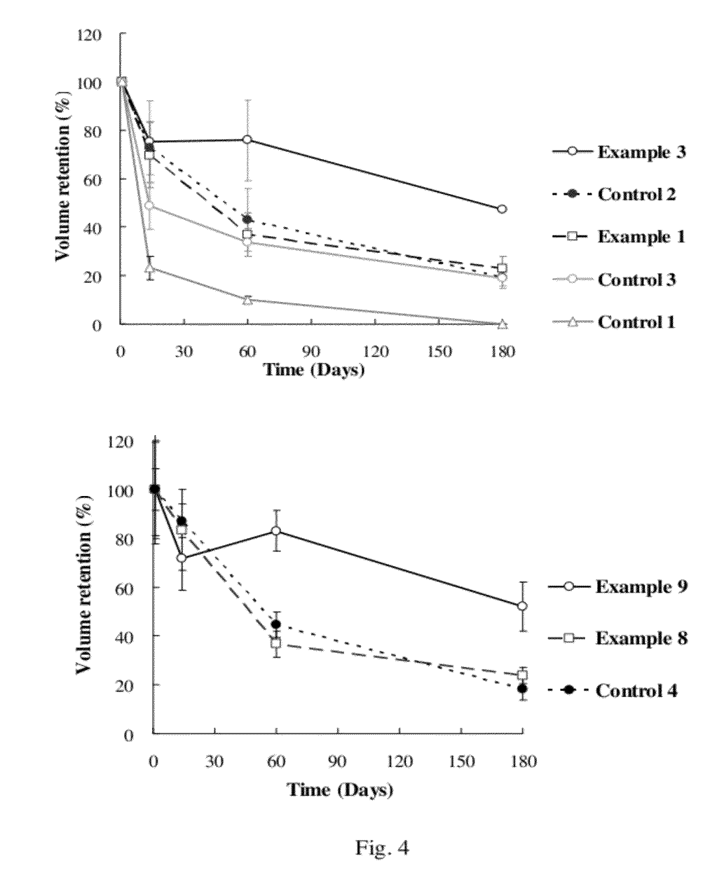

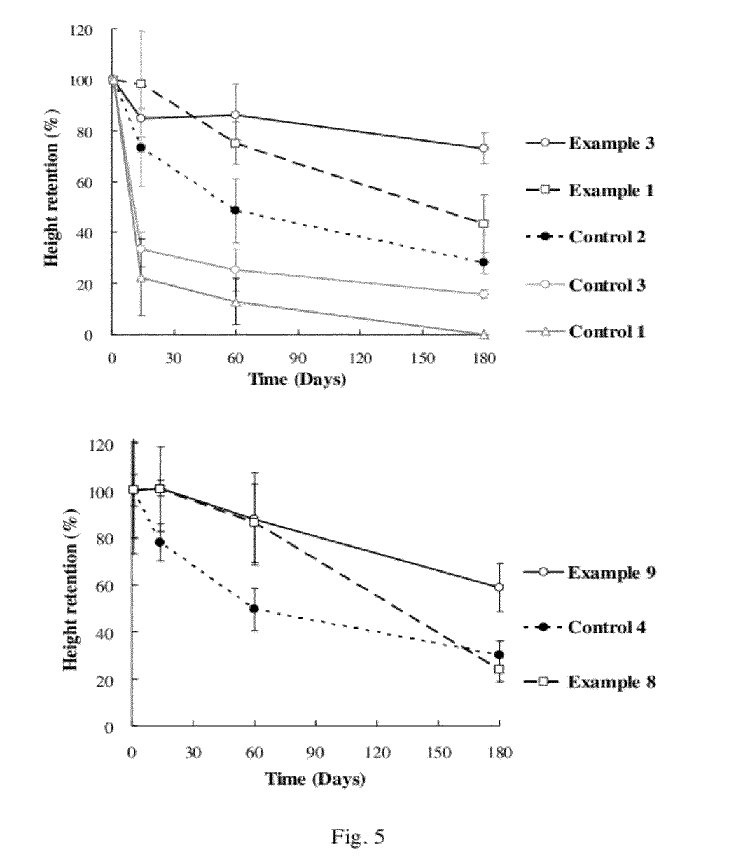

Lanyu pigs weighing between 25 and 30 kg were used in this experiment. Anesthesia was performed and the pigs lay on the operating table. 0.2 mL of each of Example 1, Example 3, Example 8, Example 9, Control 1, Control 2, Control 3 and Control 4 were subcutaneously injected into the back of the pigs' ears to form a prominence. Postoperatively, the pigs were fed regularly, and each group comprised six pigs. 8 weeks and 24 weeks postoperatively, the pigs were sacrificed for histological analysis using Masson's trichrome stain. The results were shown in FIGS. 3, 4 and 5. The histological section of the Example 3 using polyelectrolyte complex gel supported longer than Control 2 and Control 3 at each time points. The volume ratio of the Example 3 was more than 20% in the CMC groups (Control 2, Control 3) after 2 months. The height ratio of Example 3 was more than 40% in the Control 2 at 6 months.

These results show that the polyelectrolyte complex gel carrier of the present invention has excellent effect in carrying substances and cells, long degradation time and better supportability, and provides the best maintenance for soft tissue.

What is claimed is:

1. A polyelectrolyte complex gel comprising a chitosan, a γ-polyglutamic acid (γ-PGA) having a molecular weight from about 1 kDa to about 400 kDa or the salt thereof and an aqueous solution, wherein the chitosan and the γ-PGA are swollen with the aqueous solution.

2. The polyelectrolyte complex gel of claim 1, wherein the molecular weight of γ-PGA ranges from about 1 kDa to about 350 kDa, about 1 kDa to about 300 kDa, about 1 kDa to about 250 kDa, about 1 kDa to about 200 kDa, about 1 kDa to about 150 kDa, about 1 kDa to about 100 kDa, about 1 kDa to about 100 kDa, about 1 kDa to about 50 kDa, about 5 kDa to 350 kDa, about 5 kDa to about 300 kDa, about 5 kDa to about 250 kDa, about 5 kDa to about 200 kDa, about 5 kDa to about 150 kDa, about 5 kDa to about 100 kDa, about 5 kDa to about 50 kDa, about 10 kDa to about 400 kDa, about 10 kDa to about 350 kDa, about 10 kDa to about 300 kDa, about 10 kDa to about 250 kDa, about 10 kDa to about 200 kDa, about 10 kDa to about 150 kDa, about 10 kDa to about 100 kDa, about 10 kDa to about 50 kDa, about 50 kDa to about 400 kDa, about 50 kDa to about 350 kDa, about 50 kDa to about 300 kDa, about 50 kDa to about 250 kDa, about 50 kDa to about 250 kDa, about 50 kDa to about 200 kDa, about 50 kDa to about 150 kDa, about 50 kDa to about 100 kDa, about 100 kDa to about 400 kDa, about 100 kDa to about 350 kDa, about 100 kDa to about 300 kDa, about 100 kDa to about 250 kDa, about 100 kDa to about 200 kDa, or about 100 kDa to about 150 kDa, or any mixture thereof.

3. The polyelectrolyte complex gel of claim 1, wherein γ-PGA is in H form or salt form.

4. The polyelectrolyte complex gel of claim 1, wherein the salt of γ-PGA is sodium salt, potassium salt, calcium salt or magnesium salt.

5. The polyelectrolyte complex gel of claim 1, wherein the chitosan has the molecular weight more than 100 kDa.

6. The polyelectrolyte complex gel of claim 1, wherein the chitosan has the molecular weight ranging from about 100 kDa to about 2000 kDa, about 100 kDa to about 1500 kDa, about 100 kDa to about 1000 kDa, about 200 kDa to about 2000 kDa, about 200 kDa to about 1500 kDa, about 200 kDa to about 1500 kDa, about 100 kDa to about 700 kDa, about 100 kDa to about 400 kDa, or about 400 kDa to about 700 kDa, or any mixture thereof.

7. The polyelectrolyte complex gel of claim 1, wherein the amounts of the chitosan and γ-PGA or a salt thereof range from about 0.1 wt % to about 10 wt % and about 0.1 wt % to about 20 wt %.

8. The polyelectronic gel of claim 1, wherein the amount of the chitosan is about 0.5 wt % to about 10 wt %, about 1 wt % to about 10 wt %, about 2 wt % to about 10%, about 0.5 wt % to about 5 wt %, about 1 wt % to about 5 wt %, about 2 wt % to about 5 wt %.

9. The polyelectronic gel of claim 1, wherein the amount of γ-PGA or a salt thereof is about 0.5 wt % to about 20 wt %, about 1 wt % to about 20 wt %, about 1 wt % to about 15 wt %, about 1 wt % to about 10 wt % or about 1 wt % to about 5 wt %.

10. The polyelectronic gel of claim 1, which can be used as a filler or a carrier for soft tissue augmentation.

11. The polyelectronic gel of claim 1, wherein the aqueous solution has a weak acidic pH.

12. The polyelectronic gel of claim 1, wherein the aqueous solution is aqua or aqueous alcohol.

13. The polyelectronic gel of claim 12, wherein the aqueous alcohol aqua, glycerol, isopropyl alcohol, ethanol, and ethylene glycol, or mixtures thereof.

14. A soft tissue augmentation implant comprising a polyelectrolyte complex gel of claim 1 and a carrier or a filler and an optional additive.

15. The soft tissue augmentation implant of claim 14, wherein the carrier is selected from the group consisting of Acacia gel, Carbomer copolymer and homopolymer, Carbomer interpolymer, hydrogel, polysaccharide, macrocyclic polycsaccharide, oligosaccharide, starch, acetyl starch, cellulose, cellulose derivatives, methylcellulose, carboxymethylcellulose sodium, carboxymethylcellulose (CMC), ethyl (hydroxyethyl) cellulose (EHEC), ethylcellulose, hydroxypropyl cellulose, hydroxypropyl methylcellulose (HPMC), ethylcellulose, alkyl cellulose, alkoxy cellulose, hydroxy ethyl cellulose, copovidone, povidone, gelatin, Guar gum, hypromellose, hypromellose acetate succinate, maltodextrin, syrup, agar, alamic acid, aluminum monostearate, attapulgite, gellan gum, hypromellose, maltodextrin, pectin, propylene glycol alginate, sodium alginate, calcium alginate, colloidal silicon dioxide, tragacanth, xanthan gum, lecithin, tridobenzene derivatives, iohexyl, iopamidol, iopentol, sucrose, carrageenan, agarose, mannitol, saccharin sodium, sorbitol, cephalin, acetylenic diol, Carbowax, polyorgano sulfonic acid, alkoxylated surfactants, alkylphenol ethoxylates, ethoxylated fatty acids, alcohol ethoxylates, alcohol alkoxylates, polyethylene oxide, poly(propylene oxide), poly(ethylene glycol), poly(propylene glycol), poly vinyl alcohol (PVA) polymer or copolymer, polyacrylamine, poly(vinylcarboxylic acid), polymethacrylic acid, polyacrylic acid polymer or copolymer, poly amino acids, albumin, collagen, fibrin, bioglue, cellulosics, Carbopol, Poloxamer, Pluronic, Tetronics, PEO-PPO-PEO triblocks copolymer, Tetrafunctional block copolymer of PEO-PPO condensed with ethylenadiamine, polyHEMA polymer or copolymer, Hypan polymer or copolymer, starch glycolate polymer or copolymer salt, polyoxyalkylene ether, polyvinyl pyridine, polylysine, polyarginine, poly aspartic acid and poly glutamic acid, polytetramethylene oxide, poly(hydroxy ethyl acrylate), poly(hydroxy ethyl methacrylate), methoxylated pectin gels, cellulose acetate phthalate, organic oils, B-glucan, polysorbate, lactic acid ester, caproic acid ester, hyaluronic acid, dextrin, dextran, dextrose, and mixture of the above.

16. The soft tissue augmentation implant of claim 14, wherein the filler is polysaccharides (such as hyaluronic acid (HA)), inorganic salt, collagen, polyalcohols, hydroxyapatite (such as calcium hydroxyapatite), silicone and gelatin, polymethylmethacrylate or poly-L-lactic acid (PLLA), carboxymethylcellulose, cross-linked CMC hydrogel, fat or silk protein.

17. The soft tissue augmentation implant of claim 16, wherein the inorganic salt is calcium phosphate particle, calcium silicate particle, calcium carbonate particle, aluminum oxide particle, zirconium oxide particle, hydroxyapatite particle, zirconium oxide containing hydroxyapatite particle, calcium pyrophosphate particle, tetracalcium phosphate particle, tricalcium phosphate particle, octacalcium phosphate particle, fluorapatite (Ca₁₀(PO₄)₆F₂) particle, calcium apatite particle or a mixture thereof.

18. The soft tissue augmentation implant of claim 16, wherein the inorganic salt is calcium phosphate particle, zirconium oxide particle, hydroxyapatite particle, zirconium oxide containing hydroxyapatite particle or a mixture thereof.

19. The soft tissue augmentation implant of claim 16, wherein the inorganic salt is calcium phosphate particle.

20. The soft tissue augmentation implant of claim 16, wherein the inorganic salt is hydroxyapatite particle.

21. The soft tissue augmentation implant of claim 14, wherein the additive is cell, bioactive substance or protein.

22. The soft tissue augmentation implant of claim 21, wherein the cells are adipose (fat) cells, embryonic stem cells, mesenchymal stem cells, neural stem cells, preadipocytes, adipose derivated stem cells or dental pulp stem cells.

23. The soft tissue augmentation implant of claim 21, wherein the bioactive substance is a growth factor.

24. The soft tissue augmentation implant of claim 23, wherein the growth factors are epidermal growth factor (EGF), fibroblast growth factor (FGF), nerve growth factor (NGF) or a mixture thereof.
